(19) United States
(12) Patent Application Publication
(10) Pub. No.: US 2005/0005930 A1
(43) Pub. Date: Jan. 13, 2005

(54) Asthma inhalant dispenser

(76) Inventor: Phyllis Rubel, Westfield, NJ (US)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 10/831,837

(22) Filed: Apr. 26, 2004

Related U.S. Application Data
(60) Provisional application No. 60/465,084, filed on Apr. 24, 2003.

(51) Int. Cl. A61M 11/00

(52) U.S. Cl. 128/200.23; 128/200.14

(57) ABSTRACT

An asthma inhalant dispenser having a first tubular member extending in a longitudinal direction and a second tubular member connected to the first tubular member and extending in a direction transverse to the longitudinal direction of the first tubular member. The first and second tubular members are integrally connected. An attachment member may be connected to an external wall of either the first or second tubular member.

### CROSS REFERENCE TO RELATED APPLICATIONS

This application claims the benefit of U.S. Provisional Application No. 60/465,084 filed on Apr. 24, 2003, the disclosure of which is incorporated by reference herein.

### BACKGROUND OF THE INVENTION

The present invention relates to an improved asthma inhalant dispenser, and more specifically to asthma inhalant dispensers having a mechanism for attaching the dispenser to either clothes, lanyards, chains or other structures. This invention creates a means of quick and accessible portage of the inhaler, by the user, such that the user does not have to put the inhaler into a holster or pouch-type carrying device, but rather the inhaler dispenser itself is formed such that its exterior incorporates a device for attachment directly to clothing, lanyard, belt or similar structure. The design may incorporate a place for the user's personal history and identification as well as a medical emergency symbol. If a person is unable to speak or is unconscious, the fact that a medial symbol and ID are present alerts others to the victim's medical background. While the present invention improves the functionality of asthma inhalant dispensers, it does not alter methods of medication delivery or usage from the dispenser. The present invention may be used with any of the known asthma inhalant dispensers including the common Albuterol in the “L”-shaped dispenser or Advair in the newer disk-shaped dispenser.

Persons with respiratory difficulties such as asthma often need to use an inhaler to provide relief from breathing problems. The inhaler should be readily available for the user and should also be easily accessible. Instances in which an asthma inhaler should be most accessible include when a person is exercising, jogging, or participating in other aerobic activities. Ironically, it is while performing these aerobic activities that a person might not have garments which include pockets for holding their asthma inhaler or the asthma inhaler is in a pouch some distance away from the person. Consequently, it is sometimes when a person is most at risk to suffer an asthma attack that they are furthest from their medication.

Pouches or containers of various designs are known in the art for carrying articles such as medications, syringes or other articles. Examples of such devices are described in U.S. Pat. No. 6,360,929, directed to a medicinal atomizing inhaler pouch/retainer; U.S. Pat. No. 6,364,187, directed to a holster for small objects; U.S. Pat. No. 5,855,307, directed to an inhaler holster; U.S. Pat. No. 6,164,275, directed to an inhaler carrier; and U.S. Pat. No. D 453,264, directed to the design of a pouch for holding medical inhalers. Furthermore, U.S. Pat. No. D 461,239 discloses a design of an inhaler sleeve. All of these holsters/carriers suffer from the problem that they often do not allow easy accessibility to the object they are holding.

Another problem with the prior art holsters and/or carrying cases is that they increase the bulkiness of the inhaler. This result is undesirable because during aerobic activities, one would rather be free from encumbrances than be weighed down by bulky articles.

Furthermore, with the above-mentioned holster and/or carrying cases, individual canisters of medication must be placed in a dispensing unit for treatment. Consequently, if a person was to place the canister into the dispensing unit while performing aerobic activity, the cartridge may dislodge from the dispensing unit.

Accordingly, there is a need for an asthma inhalant dispenser that is quick and easy to access, minimizes the bulkiness of the dispenser unit, securely attaches to various articles, and that also securely stores an inhalant cartridge for use within the dispenser unit.

### SUMMARY OF THE INVENTION

The present invention provides an asthma inhalant dispenser having a first tubular member extending in a longitudinal direction and having a first exterior wall. The dispenser further includes a second tubular member connected to the first tubular member and having a second exterior wall. The second tubular member extends in a direction transverse to the longitudinal direction of the first tubular member. The first and second tubular members are integrally connected. The device further includes an attachment member connected to the external wall of one of the first and second tubular members.

The attachment member may be a continues loop, an elongated continuous loop, a non-continuous loop, a spring clip or a hole located in either the first or second tubular member. The asthma inhalant dispenser may also include more than one attachment member.

The dispenser may also include a locking mechanism connected to an end of the first tubular member to hold an object in a chamber defined by the first tubular member and second tubular member. The locking mechanism may include a groove located at an end of either the first or second tubular member and a plate which may be slidably received by a groove. Alternatively, the locking mechanism may also be a bar hingedly connected to an end of either the first tubular member or second tubular member with the end being remote from the other of the first or second tubular member. The bar and hinge on the locking mechanism may be integrally formed with the end of the tubular member to which it is disposed on. In still yet another alternate embodiment the locking mechanism may be a pin and rivet system.

### BRIEF DESCRIPTION OF THE DRAWINGS

A more complete appreciation of the subject matter of the present invention and the various advantages thereof can be realized by reference to the following detailed description in which reference is made to the accompanying drawings in which:

FIG. 1 is a side elevational view of an asthma inhalant dispenser having an attachment loop in accordance with a first embodiment of the present invention;

FIG. 2 is a top plan view of the asthma inhalant dispenser of FIG. 1;

FIG. 3 is a perspective view of an asthma inhalant dispenser having an attachment loop in accordance with the first embodiment of the present invention;

FIG. 4a is a side elevational view of an asthma inhalant dispenser having an attachment loop for a belt or strap in accordance with a second embodiment of the present invention;

FIG. 4b is a side elevational view of the third embodiment of the present invention; however, the strap is shown intersecting an interior surface of the dispenser at two distinct locations.

FIG. 5a is a side elevational view of an asthma inhalant dispenser having a non-continuous attachment loop in accordance with a fourth embodiment of the present invention;

FIG. 5b is a side elevational view of an asthma inhalant dispenser having a non-continuous attachment loop in accordance with the fourth embodiment of the present invention wherein the loop ends overlap one another.

FIG. 6 is a side elevational view of an asthma inhalant dispenser having a spring clip appendage in accordance with a fifth embodiment of the present invention.

FIG. 7 is a side elevational view of an asthma inhalant dispenser having a pivot clip in accordance with a sixth embodiment of the present invention.

FIG. 8 is a side elevational view of an asthma inhalant dispenser having an aperture on an exterior surface in accordance with a seventh embodiment of the present invention.

FIG. 9 is a side elevational view of the asthma inhalant dispenser of FIG. 1 with a slot and the caduceus symbol displayed on an external wall of the dispenser.

FIG. 10 is a top plan view of the asthma inhalant dispenser of FIG. 1 highlighting the slot for the medical identification card.

### DETAILED DESCRIPTION

The following discussion describes, in detail, various embodiments of the present invention. This discussion should not be construed, however, as limiting the invention to those particular embodiments. Practitioners skilled in the art will recognize numerous other embodiments as well.

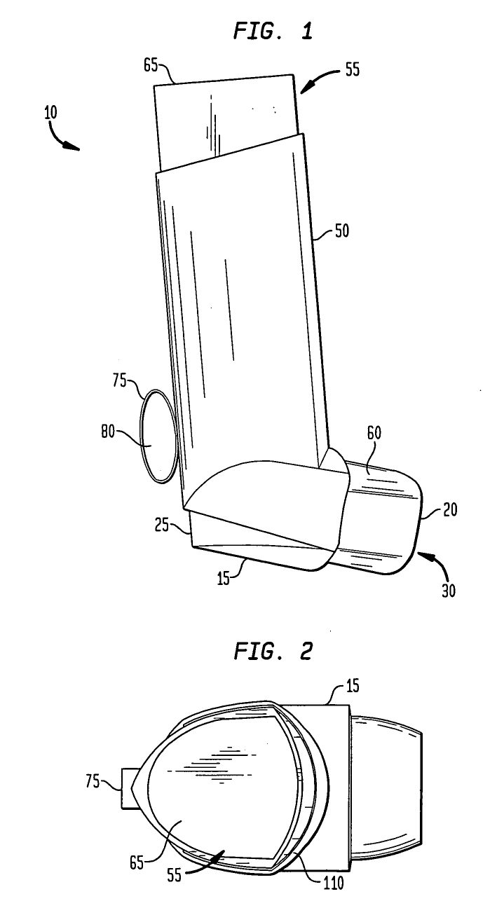

The invention is an asthma inhalant dispenser suitable for attaching to various objects via a connection mechanism. A first embodiment of the asthma inhalant dispenser 10 is shown in FIGS. 1-2. Asthma inhalant dispenser 10 includes an elongated tubular bottom portion 15 having an open end 20 providing access to an interior chamber 30, and a closed end 25. Asthma inhalant dispenser 10 further includes an elongated top portion 50, also tubular, having one end 60 connected transversely to bottom portion 15 and an open free end 65. The length of bottom portion 15 is greater than the transverse cross-section of top portion 50, so that bottom portion 15 extends radially outward beyond the periphery of top portion 50, defining a substantially L-shape. An interior chamber 55 defined in top portion 50 communicates with chamber 30 in bottom portion 15 such that, in combination, bottom portion 15 and top portion 50 define a hollow body having open ends 20 and 65. Chamber 55 is sized and shaped for receiving a canister of an asthma inhalant (not shown). Within interior chamber 30 is a nozzle (not shown) connected to a plunger (not shown). The plunger is designed so that when a canister is placed within interior chamber 55 and pushed downward, the plunger pierces a seal on the canister. This helps hold the canister in place as well as allowing the medication within the canister to exit via the nozzle when the canister is further depressed downward. Preferably, bottom portion 15 and top portion 50 have a substantially round or elliptical cross-section so as to be comfortable in the user's hand. However, other shapes, such as, rectangular, hexagonal and octagonal are also contemplated herein.

Dispenser 10 further includes an attachment member 75 connected to an exterior surface thereof. Attachment member 75 may be in the form of a continuous loop defining an aperture 80. The term “continuous loop” as used herein refers to a loop having no ends or breaks. Aperture 80 preferably is sized to accept lanyards, belts, chains, clips, etc. for use in attaching dispenser 10 to a user. Attachment member 75 may be connected to dispenser 10 by one or more fasteners, adhesives, ultrasonic welding or other known connecting techniques. Preferably, however, attachment member 75 is integrally formed with an exterior surface of dispenser 10 during a molding process. Though attachment member 75 is shown in FIG. 1 as being connected to closed end 25 of bottom portion 15, it will be appreciated by those skilled in the art that attachment member 75 may be connected to any exterior surface of dispenser 10. An advantage of varying the position of attachment member 75 on dispenser 10 is that different angles of carrying can be achieved, thus increasing the comfort of the user.

As can be best seen in FIG. 2, the bottom portion 50 of dispensing unit 10 includes a locking system 100 at open end 65. Locking system 100 acts to secure an asthma inhalant canister within chamber 55. Locking system 100 may include a strap 110 formed from a flexible material, such as a flexible polymer, leather, fabric or the like. In such event, one end of strap 110 may be fixedly connected at a position adjacent end 65 of bottom portion 50, while the other end of strap 110 may be removably connected adjacent end 65 at a position substantially diametrically opposed to the fixed connection. The fixed connection may be made by a rivet or other fastener, ultrasonic welding, adhesive, or other suitable techniques. On the other hand, the removable connection may be made by a snap, button, hook and loop fastener, or other known techniques. Alternatively, strap 110 may be formed from a rigid material, such as a rigid polymer, metal or other rigid material. In such event, one end of strap 110 may be hingedly connected adjacent end 65, in a known manner. The other end of strap 110 may be removably connected adjacent end 65, such as by a tongue-and-slot technique in which the end of the strap acts as a tongue which is removably insertable in a slot formed in bottom portion 50.

The locking system 100 may also take the form of a cap (not shown). The cap is tubular and includes an open end, a closed end and an interior chamber. The interior chamber is capable of receiving the portion of an asthma canister that extends out from the top portion of the dispenser. The open end of the cap may be screwed, snapped, hingedly connected or attached using similar techniques to the open free end of the top portion.

A person who is prone to suffer asthma attacks is able to attach asthma inhalant dispenser 10 to himself or herself through the use of a lanyard, chain, clip, articles of clothing and the like. The person can simply place a string or lanyard through aperture 80 in attachment member 75, and then tie the string or lanyard directly to the user's person, such as around their neck, wrist, arm, etc., or to an article of clothing worn by the user. Locking mechanism 100 will securely hold an asthma inhalant canister within asthma inhalant dispenser 10, even while the person wearing the dispenser is engaged in rigorous activities. The user can engage in any activity free from the worry that his or her asthma inhalant canister will escape from the asthma inhalant dispenser 10.

When a person suffers an asthma attack, he or she simply needs to untie or remove the asthma inhalant dispenser 10 from her person and place the open end 20 of bottom portion 15 in her mouth. Using a well known technique, the user may then apply an upward force to the asthma inhalant canister so as to depress a nozzle on the canister against the interior of bottom portion 15. Once the force is applied to the canister, a dose of medication is released from the canister and into the mouth of the user via chamber 30. Thus, no matter where or when an asthma attack should befall the user, the user can simply untie or slip off dispenser 10 and take her medication secure in the fact that the medication will be close at hand.

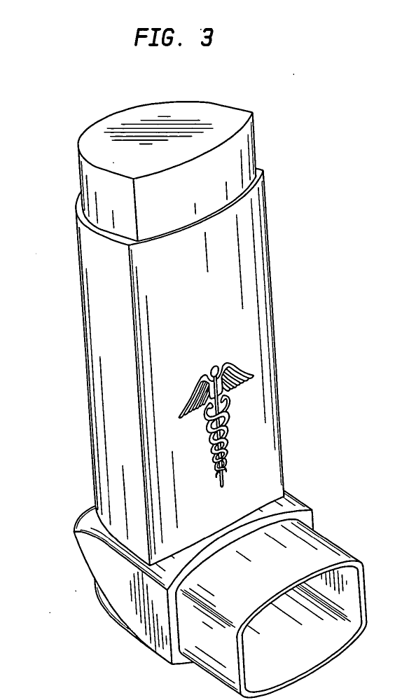

FIG. 3 is a perspective view of the first embodiment of the present invention. Attachment member 75 is located on the surface of dispenser 10. Canister 20 is shown located within interior chamber 55.

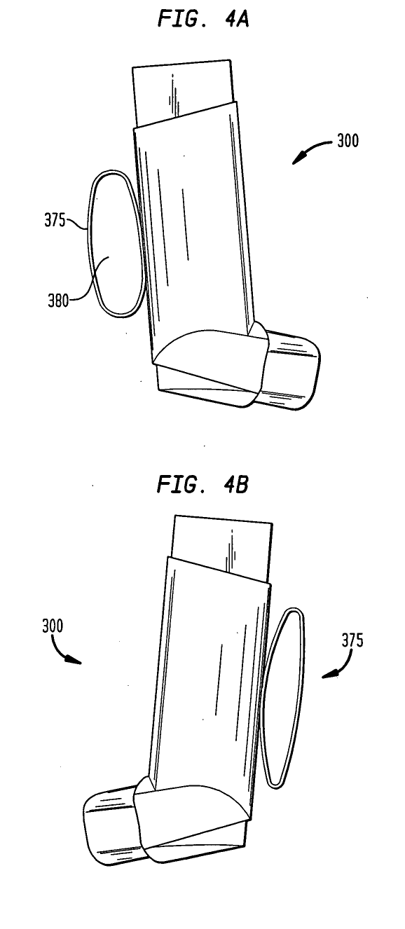

A dispenser 300 in accordance with a third embodiment of the invention is shown in FIG. 4a and 4b. Dispenser 300 is substantially the same as dispenser 10 described above. However, the attachment member is in the form of an elongated continuous loop 375 defining an elliptical aperture 380. Elliptical aperture 380 is able to receive wider support members than attachment member 75, such as belts, straps and the like. The elongated continuous loop 375 may intersect an exterior wall of dispenser 300 either at one point, as in FIG. 4a or at least at two points, as in FIG. 4b. Thus, in FIG. 4b, the elongated continuous loop 375 looks substantially like a strap connected to the dispenser.

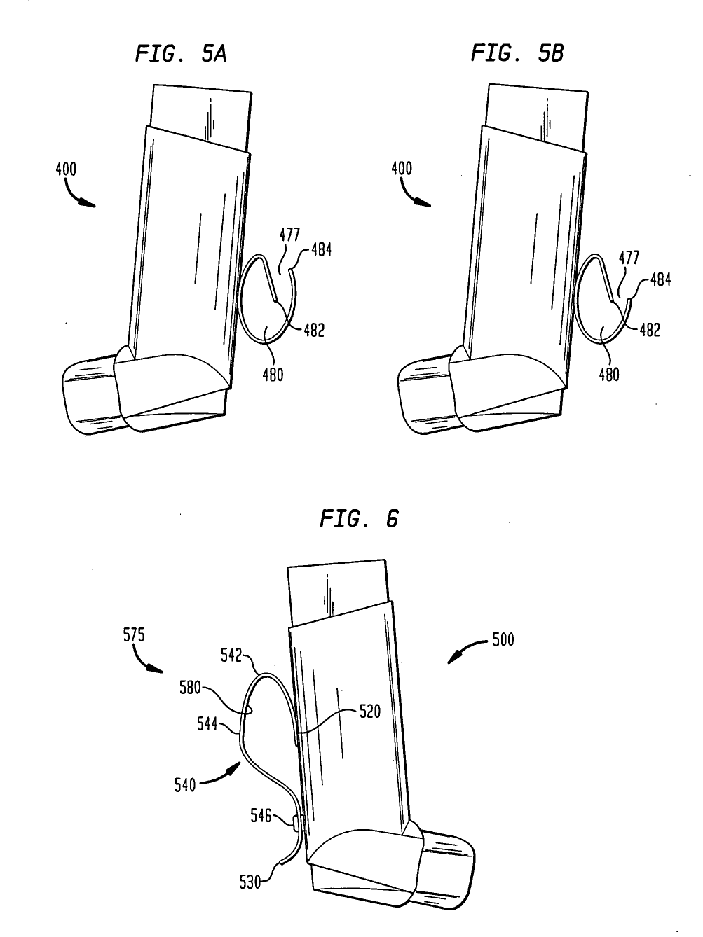

A dispenser 400 in accordance with a fourth embodiment of the invention is shown in FIGS. 5a & 5b. Once again, this embodiment works in substantially the same way as the previous embodiments; however, here the attachment member 475 is not continuous, but rather includes a slit 477 extending through the attachment member. Attachment member 475 may be cylindrical, oblong or various other shapes. The advantage of this embodiment is that the user can place a lanyard, rope or other similar support objects directly into the aperture 480 defined by attachment member 475 without having to thread the lanyard through the aperture 480. It will be appreciated that slit 477 is sufficiently narrow that the support object must be forced through the slit and into aperture 480, and therefore does not readily become disengaged, as in FIG. 5a. As shown in FIG. 5b, ends 482 and 484 may overlap one another so as to more securely lock a belt or string into aperture 480.

A dispenser 500 in accordance with a fifth embodiment of the invention is shown in FIG. 6. Dispenser 500 works in substantially the same way as the previous embodiments. Here, however, the attachment member is in the form of a spring clip 575. Spring clip 575 is made from a rigid material, such as a rigid polymer, metal or any other rigid material. Spring clip 575 has one end 520 attached to dispenser 500, and a free end 530. End 520 may be attached to dispenser 500 by a rivet or other fastener, adhesive glue, ultrasonic welding or similar connecting technique; with the preferred technique being to integrally form spring clip 575 with of dispenser 500 during a molding process.

Located between attached end 520 and free end 530 is curved member 540. Curved member 540 includes a first section 542, that curves away from dispenser 500, and a second section 544 that curves in the opposite direction back towards dispenser 500. The second section includes an end portion 546 adjacent free end 530 which again curves away from the dispenser. In a use condition, the bend between second section 544 and end portion 546 contacts dispenser 500 so as to define aperture 580. By curving away from dispenser 500, flange portion 546 enables the users to insert a lanyard, belt or support object between dispenser 500 and attachment member 575, and to face the support object into aperture 580 by flexing curved member 540 away from the dispenser.

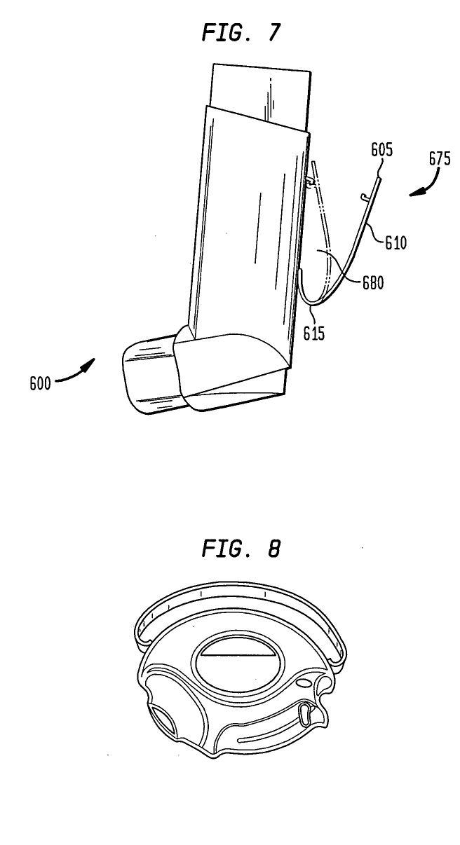

A dispenser 600 in accordance with a sixth embodiment of the invention is shown in FIG. 7. Dispenser 600 is substantially the same as the previous embodiments detailed above; however, the attachment member is in the form of a pivot clip 675. Pivot clip 675 includes a rigid member 610 pivotally connected to the exterior surface of dispenser 600. In a closed or rest position, one end 605 of rigid member 610 is spared from dispenser 600, which the other end 615 is biased into forcible contact against the dispenser. Pivot clip 600 may be pivotally rotated between the closed position, depicted in FIG. 7, and an open position in which end 615 is spaced from dispenser 500 by applying pressure to move end 605 against the dispenser. When pivot clip 675 is rotated by the user into the open position, a lanyard, belt or other such support structure may be inserted in aperture 680 located between the pivot clip and the exterior surface of the dispenser 600. After placement of the lanyard into aperture 680, pressure on end 605 may be released to rotate pivot clip 675 into the closed position whereby the lanyard is secured within the aperture.

A dispenser 700 in accordance with a seventh embodiment is shown in FIG. 8. Dispenser 700 includes an aperture 710 on an exterior surface of the dispenser, capable of receiving a chain, string or similar structure. The chain or string might include an end portion that is capable of entering aperture 710, but once within the dispenser it is locked within. Furthermore, in an embodiment not shown, dispenser 700 may include a plurality of apertures on its exterior surface. Multiple apertures would allow a user to thread a chain or string through one aperture and out another. The dispenser may then be tied to the user's person or other objects via the chain or string.

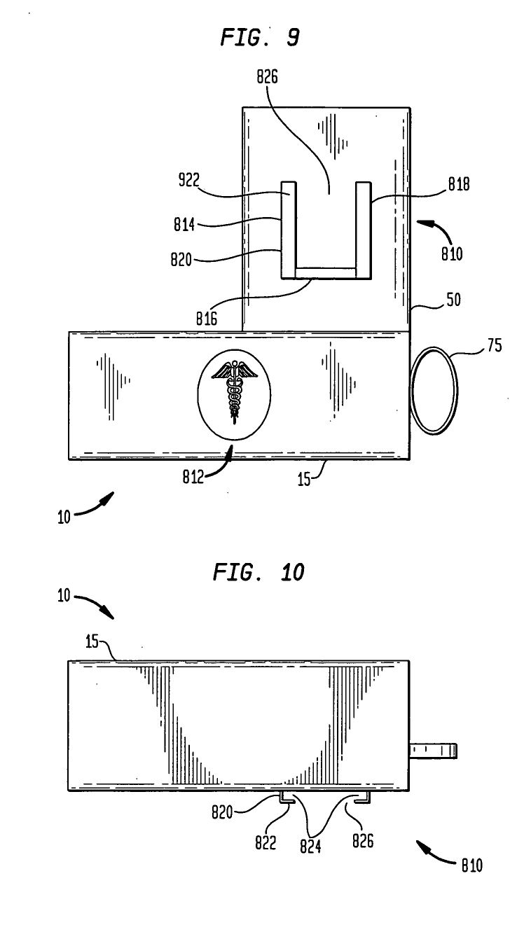

The first embodiment of dispenser 10 is again shown in FIG. 9 and FIG. 10 and includes medical identification slot 810 and medical symbol “caduceus” 812. The caduceus symbol 812 may be in the form of a decal, sticker, printing, a separately molded symbol or any other device attached to the exterior of dispenser 10. In a preferred embodiment, caduceus symbol 812 is integrally molded as either a positive or negative feature with either the top or bottom portion of the dispenser. Though dispenser 10 is described here in conjunction with the caduceus symbol, any medical symbol may be included with the dispenser so as to alert the public to the importance of the dispenser. Medical identification slot 810 includes a first rail 814, a second rail 816 and a third rail 818. Rail 814 extends longitudinally with top portion 50 and includes first wall 820 extending outwardly from top portion 50 and integrally attached along one edge to dispenser 10. A second wall 822 is adjacent to first wall 820 and integrally attached to first wall 820 opposite the dispenser 10. Dispenser 10, first wall 820 and second wall 822 define groove 824. Rail 816 is designed and connected to dispenser 10 in substantially the same way as rail 814. However, rail 816 is integrally connected to the dispenser so as to be perpendicular to rail 814. Rail 818 is also designed and integrally connected to dispenser 10 in substantially the same way as rail 814. Rail 818 is integrally connected to the dispenser so as to be perpendicular to rail 816 opposite and spaced from rail 814. Rail 818 extends upwardly from rail 816 by about the same amount as rail 814. Groove 824 extends from rail 814 to rail 816 to rail 818 so as to define slot 826. Slot 826 is capable of receiving a medical identification card (not shown) through its open end. The medical identification card can then be slidably received within slot 826 so that the card is in contact with and held in place by all three rails.

In alternative embodiments the medical ID display may be a decal or sticker attached to the exterior of the dispenser. The medical ID display may include personal information about the user, such as name or address. This feature enables the user and others to identify the owner of the dispenser in case it is misplaced or lost.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An asthma inhalant dispenser, comprising:
a first tubular member extending in a longitudinal direction and having a first exterior wall;
a second tubular member connected to said first tubular member and having a second exterior wall and extending in a direction transverse to the longitudinal direction, wherein said first and second tubular members are integrally connected; and
an attachment member connected to said external wall of one of said first and second tubular members.

2. The asthma inhalant dispenser of claim 1 wherein said attachment member is a continuous loop.

3. The asthma inhalant dispenser of claim 2 wherein there is more than one continuous loop.

4. The asthma inhalant dispenser of claim 1 wherein said attachment member is an elongated continuous loop.

5. The asthma inhalant dispenser of claim 1 wherein said attachment member is a non-continuous loop.

6. The asthma inhalant dispenser of claim 1 wherein said attachment member is a spring clip.

7. The asthma inhalant dispenser of claim 1 wherein said attachment member is a hole located in either said first or said second tubular member.

8. The asthma inhalant dispenser according to claim 1 wherein said attachment member is molded integrally with either said first or said second tubular member.

9. The asthma inhalant dispenser according to claim 1 further comprising a chamber wherein a locking mechanism is connected to a lower end of said first tubular member to hold an object in said chamber.

10. The asthma inhalant dispenser as in claim 9, further comprising a groove and a plate wherein said locking mechanism is defined by said groove at an end of either said first tubular member or said second tubular member, said end being remote from said other of said first or said second tubular member, said groove extending partially circumferentially around said end of said first or second tubular member and said plate being capable of sliding in and out of said groove.

11. The asthma inhalant dispenser as in claim 9, wherein said locking mechanism is a bar hingedly connected to an end of either said first tubular member or said second tubular member, said end being remote from said other of said first or second tubular member.

12. The asthma inhalant dispenser as in claim 10, wherein said locking mechanism is a bar hingedly connected to an end of either said first tubular member or said second tubular member, said end being remote from said other of said first or second tubular member.

13. The asthma inhalant dispenser as in claim 12, wherein said bar and hinge are integrally formed with said end of either said first tubular member or said second tubular member.

14. The asthma inhalant dispenser as in claim 9, wherein said locking mechanism is defined by a pin/rivet system located at an end of either said first tubular member or said second tubular member, said end being remote from said other of said first or second tubular member.

15. The asthma inhalant dispenser as in claim 1, further comprising a medial symbol or personal identification insignia disposed on said exterior wall of either said first or said second tubular member.
